(19) United States
(12) Patent Application Publication
(10) Pub. No.: US 2026/0202361 A1
(43) Pub. Date: Jul. 16, 2026

(54) MITIGATING PET TOF RECONSTRUCTION INCONSISTENCY FOR A PET SCANNER WITH BGO-BASED DETECTORS CONFIGURED TO DETECT CHERENKOV PHOTONS

(71) Applicant: GE Precision Healthcare LLC, Waukesha, WI (US)

(72) Inventors: Raz Carmi, Haifa (IL); Danielle Zeltser, Haifa (IL); Yariv Grobshtein, Haifa (IL)

(73) Assignee: GE Precision Healthcare LLC, Waukesha, WI (US)

(21) Appl. No.: 19/019,619

(22) Filed: Jan. 14, 2025

(51) Int. Cl. G01N 23/2202

(52) U.S. Cl. CPC G01N 23/2202; G01N 2201/121

(57) ABSTRACT

A PET imaging system includes BGO crystals that generate Cherenkov photons in response to excitation by photons from positron annihilation events and photosensors configured to detect the Cherenkov photons and generate signals indicative thereof, an LOR determiner configured to identify, based on the signals, coincident photon pairs along each LOR, a TOF determiner configured to determine a timing resolution for each of the coincident photon pair along each LOR, an accuracy determiner configured to bin the LORs, based on the timing resolutions, into at least higher accuracy TOF LORs and lower accuracy TOF LORs, a reconstructor configured to reconstruct the higher accuracy TOF LORs using models and generate volumetric image data, and a model adjuster configured to adjust at least one model of the models based on the volumetric image data. The reconstructor is configured to reconstruct at least a subset of the LORs using the updated models.

### FIELD

The following generally relates to Positron Emission Tomography (PET) and more particularly to mitigating PET Time-Of-Flight (TOF) reconstruction inconsistency for a PET scanner that includes detectors with Bismuth Germanate (Bi4Ge3O12, BGO) scintillating crystals and configured to detect Cherenkov photons.

### BACKGROUND

Positron Emission Tomography (PET) is a functional imaging modality that utilizes a radiopharmaceutical with a tissue targeted radionuclide (i.e., a radiotracer) to visualize and/or measure functional processes such as metabolism, blood flow, absorption, etc. Prior to a PET scan, a radiopharmaceutical is administered to a patient. As the radionuclide accumulates within organs, vessels, or the like, the radionuclide undergoes positron emission decay and emits a positron. When the positron collides with an electron in the surrounding tissue, both the positron and the electron are annihilated and converted into a pair of 511 keV photons (i.e., gamma rays).

The two 511 keV photons are directed in substantially opposite directions along a line of response (LOR) and are coincidently detected when they reach respective detectors positioned across from each other on a detector ring assembly, approximately one hundred and eighty degrees apart from each other. When the 511 keV photons impinge upon scintillation crystals of the detectors, a scintillation event (e.g., a flash of light) is produced for each 511 keV photon, and detectors detect the scintillation photons and produce electrical signals indicative thereof. The electrical signals are processed to generate PET data, which represent a distribution of the radiopharmaceutical within the patient, which may be employed to observe metabolic processes, etc. in the body and diagnose disease.

PET image reconstruction algorithms include Time-of-Flight (TOF)-based reconstruction algorithms and non-TOF reconstruction algorithms. In general, when a positron annihilation event occurs closer to a one detector crystal than the opposing detector crystal, a 511 keV photon impinges the closer detector crystal before (e.g., nanoseconds or picoseconds before) the other 511 keV photon impinges the other detector crystal. The TOF (i.e., timing) difference between the detections of the 511 keV photon allows for an estimation of a location of the positron annihilation event along the LOR. Utilizing TOF information in PET image reconstruction improves image quality.

PET detector crystals have included Bismuth Germanate (Bi4Ge3O12, BGO)-based detector crystals and Lutetium-based detector crystals, such as cerium-doped lutetium oxy-orthosilicate (Lu2SiO5(Ce), LSO) and lutetium-yttrium oxy-orthosilicate (Lu1.8Y0.2SiO5(Ce), LYSO). BGO has a higher effective atomic number and density relative to Lutetium-based detector crystals, which makes it more effective at stopping gamma rays, leading to higher detection efficiency. BGO also has a lower intrinsic background radiation relative to Lutetium-based detector crystals, which can improve the signal-to-noise ratio of the PET volumetric image data. BGO detector crystals are also generally less expensive than Lutetium-based detector crystals.

However, BGO has a longer scintillation decay time (i.e., a lower timing resolution), which limits its accuracy, relative to Lutetium-based detector crystals, and the advantages provided by TOF information depend on the timing resolution of the detector crystals. BGO detector crystals also produce Cherenkov photons in response to interactions with the 511 keV photons, which are faster than BGO scintillation photons. The literature indicates combining BGO detector crystals with fast silicon photomultipliers (SiPMs) and detecting and utilizing Cherenkov photons provides for improving the timing resolution, making them competitive with Lutetium-based detector crystals for TOF-based PET imaging.

The timing resolution for the Cherenkov photons varies from annihilation event to annihilation event, and only a small fraction of the LORs will have an effective timing resolution comparable to the timing resolution of Lutetium-based detector crystals. The literature indicates that the variable timing resolution can be determined, and the LORs can then be classified based on timing resolution, where each group of LORs corresponds to a different timing resolution range (i.e., accuracy), and then the PET data can be reconstructed using techniques that integrate the variable timing resolutions within the PET image reconstruction iterations using different kernels.

In order to reconstruct diagnostic quality PET volumetric image data, the PET data is corrected for random coincidences, photon attenuation, Compton scattering of photons in tissue, patient motion, etc. The accuracy of these corrections strongly affects the image quality of the final reconstructed PET volumetric image data. Although TOF-based reconstruction is less sensitive to inconsistencies between the PET data and the corrections than non-TOF-based reconstruction, TOF-based reconstruction nevertheless is sensitive to such inconsistencies, which manifest as artifact and degrade image quality.

In view of the foregoing, there is an unresolved need for an approach that mitigates such inconsistencies with TOF based reconstructions for PET scanners with BGO-based detectors configured to detect Cherenkov photons.

### SUMMARY

Aspects described herein address the above-referenced problems and others. This summary introduces concepts that are described in more detail in the detailed description. It should not be used to identify essential features of the claimed subject matter, nor to limit the scope of the claimed subject matter.

In one aspect, a PET imaging system includes Bismuth Germanate (BGO) crystals that generate Cherenkov photons in response to excitation by photons from positron annihilation events and photosensors configured to detect the Cherenkov photons and generate signals indicative thereof. The PET imaging system further includes a line of response (LOR) determiner configured to identify, based on the signals, coincident photon pairs along each LOR. The PET imaging system further includes a time-of-flight (TOF) determiner configured to determine a timing resolution for each of the coincident photon pair along each LOR. The PET imaging system further includes an accuracy determiner configured to bin the LORs, based on the timing resolutions, into at least higher accuracy TOF LORs and lower accuracy TOF LORs. The PET imaging system further includes a reconstructor configured to reconstruct the higher accuracy TOF LORs using models and generate volumetric image data. The PET imaging system further includes a model adjuster configured to adjust at least one model of the models based on the volumetric image data. The reconstructor is configured to reconstruct at least a subset of the LORs using the updated models.

In another aspect, a computer-implemented method includes receiving signals from photosensors of a PET imaging system. The signals are indicative of Cherenkov photons produced by BGO crystals in response to excitation by photons from positron annihilation events directed in substantially opposite directions along LORs. The method further includes identifying, based on the signals, coincident photons along each LOR. The method further includes determining a timing resolution for each identified pair of coincident photons. The method further includes binning the LORs, based on the timing resolutions, into at least higher accuracy TOF LORs and lower accuracy TOF LORs. The method further includes reconstructing the higher accuracy TOF LORs using models to generate volumetric image. The method further includes adjusting at least one model of the models based on the volumetric image. The method further includes reconstructing at least a subset of the LORs using the updated models.

In another aspect, a computer readable storage medium includes computer readable instructions, which when executed by a computer processor, causes the computer processor to receive signals from photosensors of a PET imaging system. The signals are indicative of Cherenkov photons produced by BGO crystals in response to excitation by photons from positron annihilation events directed in substantially opposite directions along LORs. The instructions further cause the computer processor to identify, based on the signals, coincident photons along each LOR. The instructions further cause the computer processor to determine a timing resolution for each identified pair of coincident photons. The instructions further cause the computer processor to bin the LORs, based on the timing resolutions, into at least higher accuracy TOF LORs and lower accuracy TOF LORs. The instructions further cause the computer processor to reconstruct the higher accuracy TOF LORs using models to generate volumetric image. The instructions further cause the computer processor to adjust at least one model of the models based on the volumetric image. The instructions further cause the computer processor to reconstruct at least a subset of the LORs using the updated models.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings in which like references indicate similar elements.

FIG. 1 schematically illustrates a cross-sectional side view of an example multi-modality imaging system with functional and anatomical imaging capabilities and an inconsistency mitigation module, in accordance with an embodiment(s) herein.

FIG. 2 schematically illustrates a front view of a non-limiting example of a PET functional imaging system sub-system of the multi-modality imaging system of FIG. 1, in accordance with an embodiment(s) herein.

FIG. 3 schematically illustrates a front view of a non-limiting example of a CT anatomical imaging system sub-system of the multi-modality imaging system of FIG. 1, in accordance with an embodiment(s) herein.

FIG. 4 schematically illustrates an example of the inconsistency mitigation module using only higher accuracy TOF PET data to update models, in accordance with an embodiment(s) herein.

FIG. 5 schematically illustrates another variation of the example inconsistency mitigation module described in connection with FIG. 4 that further employs lower accuracy TOF PET data to update the models, in accordance with an embodiment(s) herein.

FIG. 6 depicts example PET volumetric image data reconstructed without employing the inconsistency mitigation module.

FIG. 7 depicts example high-consistent, low-statistic correction volumetric image data using the inconsistency mitigation module, in accordance with an embodiment(s) herein.

FIG. 8 depicts example PET volumetric image data reconstructed using the correction volumetric image generated by the inconsistency mitigation module, in accordance with an embodiment(s) herein.

FIG. 9 schematically illustrates an example of the models, in accordance with an embodiment(s) herein.

FIG. 10 illustrates a non-limiting example of a flow chart for a computer-implemented method for mitigating reconstruction inconsistencies for a BGO-PET based TOF reconstruction using only corrected higher accuracy TOF data to update the models, in accordance with an embodiment(s) herein.

FIG. 11 illustrates a non-limiting example of a flow chart for a computer-implemented method for mitigating reconstruction inconsistencies for a BGO-PET based TOF reconstruction using corrected higher accuracy TOF data and reconstructed uncorrected higher accuracy TOF data to update the models, in accordance with an embodiment(s) herein.

FIG. 12 illustrates a non-limiting example of a flow chart for a computer-implemented method for mitigating reconstruction inconsistencies for a BGO-PET based TOF reconstruction using corrected higher accuracy TOF data and corrected lower accuracy TOF data to update the models, in accordance with an embodiment(s) herein.

FIG. 13 illustrates a non-limiting example of a flow chart for a computer-implemented method for mitigating reconstruction inconsistencies for a BGO-PET based TOF reconstruction using corrected higher accuracy TOF data, reconstructed uncorrected higher accuracy TOF data, and corrected lower accuracy TOF data to update the models, in accordance with an embodiment(s) herein.

FIG. 14 schematically illustrates a cross-sectional side view of another example multi-modality imaging system with functional and anatomical imaging capabilities and the inconsistency mitigation module, in accordance with an embodiment(s) herein.

### DETAILED DESCRIPTION

Embodiments of the present disclosure will now be described, by way of example, with reference to the figures, in which a system, a method and/or a computer readable medium includes instructions for mitigating Positron Emission Tomography (PET) Time-Of-Flight (TOF)-based reconstruction inconsistency for a PET scanner that includes detectors with Bismuth Germanate (Bi4Ge3O12, BGO) detector crystals and photosensors configured to detect events related to at least Cherenkov photons. Such inconsistencies can be associated with one or more of attenuation correction, scatter correction, motion-phase matching, body-contouring, multi-modality registration, prior-based regularized reconstruction, and/or other inconsistencies that degrade image quality, etc., and at least a certain subset of the detected Cherenkov photons can be employed to update and/or improve models employed during reconstruction to mitigate such inconsistencies, which improves image quality.

As described in greater detail below, in one instance the approach herein utilizes only detected Cherenkov photons with timing resolutions satisfying predetermined timing resolution criteria (i.e., higher accuracy TOF LORs) to reconstruct high-consistent, low-statistic volumetric image data. This includes employing one or more of the models in the set of models (before the models are updated) to reconstruct the higher accuracy TOF LORs and generate the high-consistent, low-statistic volumetric image data, employing the higher-consistent, lower-statistic volumetric image data to update and/or improve one or more of the models, and then using the updated and/or improved models to reconstruct PET data and generate volumetric image data. In one instance, this PET data includes an entirety of the LORs (i.e., higher accuracy TOF LORs and the remaining TOF LORs), only the higher accuracy LORs, and/or other PET data.

In some instances, the higher accuracy TOF LORs are also reconstructed without using any of the models to generate additional volumetric image data, and both higher-consistent, lower-statistic volumetric image data and the additional volumetric image data are utilized to update and/or improve one or more of the models. In some instances, the lower accuracy TOF LORs are also reconstructed but without using any of the models to generate other volumetric image data, and both higher-consistent, lower-statistic volumetric image data and the other volumetric image data are utilized to update and/or improve one or more of the models. In some instances, both the additional volumetric image data and the other volumetric image data are reconstructed and employed along with the higher-consistent, lower-statistic volumetric image data to update and/or improve one or more of the models.

In one instance, the approach described herein mitigates inconsistencies (e.g., patient relate, data matching, data registration, etc.), such as one or more inconsistencies associated with one or more of attenuation correction, scatter correction, motion-phase matching, body-contouring, multi-modality registration, prior-based regularized reconstruction, etc., improving image quality relative to a configuration that does not employ the approach herein. For example, the image quality is improved to a predetermined level for a specific clinical diagnostic purpose, to a level corresponding to no or little inconsistency, etc. The improved image quality can be expressed for example, besides higher spatial resolution and lower image noise, in reduced structural image artifacts, higher contrast to noise ratio, or more accurate quantification values. In one instance, this is achieved while maintaining the spatial resolution within a range such that the spatial resolution of the PET volumetric image data after employing the approach described herein is at least at a level of the spatial resolution of PET volumetric image data reconstructed without employing the approach described herein.

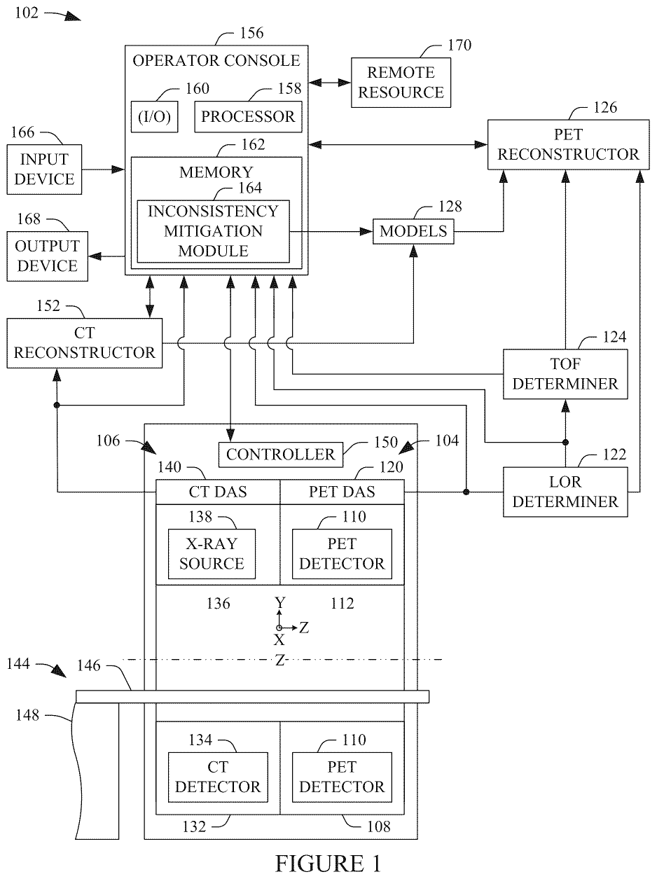

Referring initially with FIG. 1, a cross-sectional side view of a multi-modality imaging system 102 with functional and anatomical imaging capabilities is schematically illustrated. In general, the imaging system 102 includes a functional imaging sub-system 104 and an anatomical imaging sub-system 106 integrated together in a single imaging system. In this example, the functional imaging sub-system 104 is configured for PET imaging and the anatomical imaging sub-system 106 is configured for Computed Tomography (CT) imaging. In another instance, the anatomical imaging sub-system 106 is configured for Magnetic Resonance (MR) and/or other anatomical imaging. In another instance, the functional imaging sub-system 104 and the anatomical imaging sub-system 106 are part of different imaging systems (e.g., separate PET and CT, MR, etc. scanners).

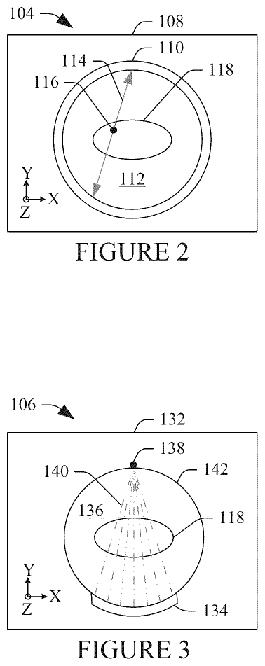

Briefly turning to FIG. 2, an example front view of the PET imaging sub-system 104 is schematically illustrated. With reference to FIGS. 1 and 2, the PET imaging sub-system 104 includes a PET gantry 108. The PET gantry 108 includes a radiation sensitive detector array 110 disposed in a generally annular ring about a PET bore 112. The radiation sensitive detector array 110 includes a plurality of detectors with a plurality of detector crystals in optical communication with a plurality of photosensors, where the plurality of detector crystals is disposed between the plurality of the photosensors and the PET bore 112.

The detector crystals include a material that produces Cherenkov photons in response to excitation by 511 keV photons 114 (FIG. 2) produced in response to a positron annihilation event 116 (FIG. 2) occurring in the PET bore 112 in a patient 118 (FIG. 2) disposed therein. An example of such a material is Bismuth Germanate (Bi4Ge3O12, BGO) and/or other material. The plurality of photosensors convert the Cherenkov photons into electrical signals. An example of a suitable photosensor includes a silicon photomultipliers (SiPMs), such as a fast SiPM with low single photon time resolution (SPTR) values combined with fast readout electronics, and/or other photosensor.

With reference to FIG. 1, the PET imaging sub-system 104 further includes a PET data acquisition system (DAS) 120. The PET data acquisition system 120 receives the signals from the radiation sensitive detector array 110 and produces PET emission data, which includes a list of events detected by the plurality of radiation sensitive detectors 110. A LOR determiner 122 is configured to identify coincident gamma pairs by identifying events detected in temporal coincidence (or near simultaneously) along a line of response (LOR), which is a straight line joining the two detectors detecting the events and generates list mode data and/or a histogram (sinogram) indicative thereof.

Coincidence can be determined by a number of factors, including event time markers, which must be within a predetermined time period of each other to indicate coincidence, and the LOR. Events that cannot be paired can be used to estimate and correct random coincidences, but are not directly used in the reconstructed data. Events that can be paired are located and recorded as coincidence event pairs. The PET emission data provides information on the LOR for each event, such as a transverse position and a longitudinal position of the LOR and a transverse angle and an azimuthal angle. Additionally, or alternatively, the PET emission data is re-binned into one or more sinograms or projection bins.

A TOF determiner 124 is configured to determine Time-Of-Flight (TOF) information for the LORs. Again, the TOF information allows for estimating a location of an event along a LOR. For example, when a positron annihilation event occurs closer to a first detector crystal than a second detector crystal, one of the annihilation photons reaches the first detector crystal before (e.g., nanoseconds or picoseconds) the other annihilation photon reaches the second detector crystal. The TOF difference of the two photons at their respective detector crystals is utilized to constrain a location of the positron annihilation event along the LOR.

The timing resolution for the Cherenkov photons varies from annihilation event to annihilation event, e.g., between an effective one hundred and eighty (180) picoseconds (ps) to eight hundred (800) ps, where a smaller value corresponds to higher accuracy, and only a small fraction of the LORs (e.g., ~10-20 %) will have an effective timing resolution comparable to the timing resolution of Lutetium-based detector crystals. As utilized herein, the term higher-accuracy TOF LORs refers to LORs with shorter timing resolutions (i.e., smaller values), and the term lower-accuracy TOF LORs refers to LORs with relatively higher timing resolutions (i.e., larger values). The timing resolution for each coincidence event pair along a LOR is determined by the TOF determiner 124, as part of the determined TOF information. This can be done for example, per event, by analyzing the signal characteristics of the Cherenkov photons relative to the signal characteristics of the scintillation photons.

A PET reconstructor 126 reconstructs PET data (e.g., LORs with or without the TOF data) using known iterative or other techniques and employing at least one model of a set of models 128 to generate PET volumetric image data indicative of the distribution of the radionuclide in a scanned subject. In one instance, the set of models 128 includes models corresponding to one or more of attenuation correction, scatter correction, motion-phase matching, body-contouring, multi-modality registration, prior-based regularized reconstruction, etc. Suitable reconstruction algorithms include an ART technique, an analytic image reconstruction algorithm such as FBP, etc., an iterative image reconstruction algorithm such as Ordered Subset Expectation Maximization (OSEM), a Block Sequential Regularized Expectation Maximization (BSREM) algorithm, etc., another algorithm and/or a combination thereof.

Briefly turning to FIG. 3, an example front view of the CT imaging sub-system 106 is schematically illustrated. With reference to FIGS. 1 and 3, the CT imaging sub-system 106 includes a CT gantry 132. The CT gantry 132 includes a detector array 134 disposed about an isocenter of a CT bore 136 along an annular ring. The CT gantry 132 further includes an X-ray source 138, such as an X-ray tube or source, that rotates about the CT bore 136. The detector array 134 detects radiation 140 (FIG. 3) emitted by the radiation source 138 that has traversed the CT bore 136 and the subject 118 (FIG. 3) therein.

The X-ray source 138 and the detector array 134 are disposed on a rotating frame 142 (FIG. 3), opposite each other, across the CT bore 136. The rotating frame 142 rotates the X-ray source 138 in coordination with the detector array 134. The X-ray source 138 emits the X-ray radiation 140, which traverses the CT bore 136 and the subject 118 disposed therein, and the detector array 134 detects X-ray radiation impingent thereon. For each arc segment, the detector array 134 generates a view of projections. A CT data acquisition system (DAS) 140 (FIG. 1) processes the signals from the detector array 134 to generate projection data indicative of the radiation attenuation along a plurality of lines or rays through the CT bore 136.

With reference to FIG. 1, a table 144 includes a cradle 146 moveably coupled to a frame/base 148. In one instance, the cradle 146 is slidably coupled to the frame/base 148 via a bearing or the like, and a drive system (not visible) including a controller, a motor, a lead screw, and a nut (or other drive system) translates the cradle 146 along the frame/base 148 into and out of the CT bore 136 and/or the PET bore 112. The cradle 146 is configured to support the subject 118 in the CT bore 136 and/or the PET bore 112 for loading, scanning, and/or unloading the subject. The CT bore 136 and/or the PET bore 112 are disposed along a common longitudinal or z-axis (Z). Where the PET and CT sub-systems 104 and 106 are separate imaging systems, with each having its own table.

Continuing with FIG. 1, a controller 150 is configured to control components such as rotation of the rotating frame 142 (FIG. 3), an operation of the X-ray source 138, an operation of the detector arrays 134 and/or 110, an operation of the table 144, etc. For example, in one embodiment the controller 150 includes a table controller configured to control motion and/or height of the table 144 for loading, scanning and/or unloading the subject or object. Where the PET and CT sub-systems 104 and 106 are separate imaging systems, each can have its own controller.

Continuing with FIG. 1, a CT reconstructor 152 reconstructs the CT projection data using known iterative or other techniques to generate volumetric image data (i.e., CT image data) indicative of the radiation attenuation of the subject or object. Suitable reconstruction algorithms include an algebraic reconstruction technique (ART), an analytic image reconstruction algorithm such as filtered backprojection (FBP), etc., an iterative reconstruction algorithm such as advanced statistical iterative reconstruction (ASIR), a maximum likelihood expectation maximization (MLEM) algorithm, etc., another algorithm and/or a combination thereof.

The operator console 156 further includes a processor 158 such as a central processing unit (CPU), a graphics processing unit (GPU), a micro-processing unit (μPU), etc., and input/output (I/O) 160. The operator console 156 further includes a computer readable storage medium 162 (“MEMORY”), which includes non-transitory medium (e.g., a storage cell, a device, etc.) and excludes transitory medium (i.e., signals, carrier waves, and the like). In the illustrated example, the operator console 156 receives one or more of CT projection data, CT image data, a CT attenuation map, PET emission data, PET projections, PET list mode data, PET LORs, a PET sinogram, PET TOF information, etc.

The memory 162 is encoded with computer-executable instructions. In the illustrated example, the computer-executable instructions include an inconsistency mitigation module 164 configured to mitigate PET TOF-based reconstruction inconsistency for the imaging subsystem 102 (which includes BGO-based detectors configured to detect Cherenkov photons), including inconsistencies associated with one or more of attenuation correction, scatter correction, motion-phase matching, body-contouring, multi-modality registration, prior-based regularized reconstruction, etc.

As described in greater detail below, in one instance the approach herein utilizes only the higher accuracy TOF LORs along with one or more of the models 128 to reconstruct high-consistent, low-statistic volumetric image data that is utilized to update and/or improve one or more of the models 128. In some instances, the approach further utilizes the higher accuracy TOF LORs without any of the models 128 to generate additional volumetric image data and/or the lower accuracy TOF LORs along with one or more of the models 128 to generate other volumetric image data, where the additional and/or the other volumetric image data is further utilized to update and/or improve one or more of the models 128.

Again, the approach described herein mitigates inconsistencies such as one or more inconsistencies associated with one or more of attenuation correction, scatter correction, motion-phase matching, body-contouring, multi-modality registration, prior-based regularized reconstruction, etc., improving image quality relative to a configuration that does not employ the approach herein, and while maintaining the spatial resolution within a range such that the spatial resolution of the PET volumetric image data after employing the approach described herein is at least at a level of the spatial resolution of PET volumetric image data reconstructed without employing the approach described herein.

Returning to FIG. 1, the imaging system 102 further includes an operator console 156. The operator console 156 includes a computing system such as a computer, a workstation, a server, or the like. The operator console 156 includes an input device 166 such as a keyboard, mouse, touchscreen, microphone, etc., and an output device 168 such as a human readable device such as a display monitor or the like. The (I/O) 160 is configured for transmitting and/or receiving signals and/or data, e.g., via the input device 166, output device 168, wireless technology, portable devices, etc.

The imaging system 102 further includes a remote resource 170. In one instance, the remote resource 170 includes a radiology information system (RIS), a hospital information system (HIS), an electronic medical record (EMR), a picture archiving and communication system (PACS), one or more other individual and/or hybrid imaging systems, a server, a database, a cloud-based resource (including shared remote data storage and/or computing power, including processing resources distributed over multiple locations/data centers), etc. The imaging system 102 is in electrical communication with the remote resource 166 and is configured to transmit and/or receive image data via Digital Imaging and Communications in Medicine (DICOM), etc., and other data via Health Level Seven (HL7), etc.

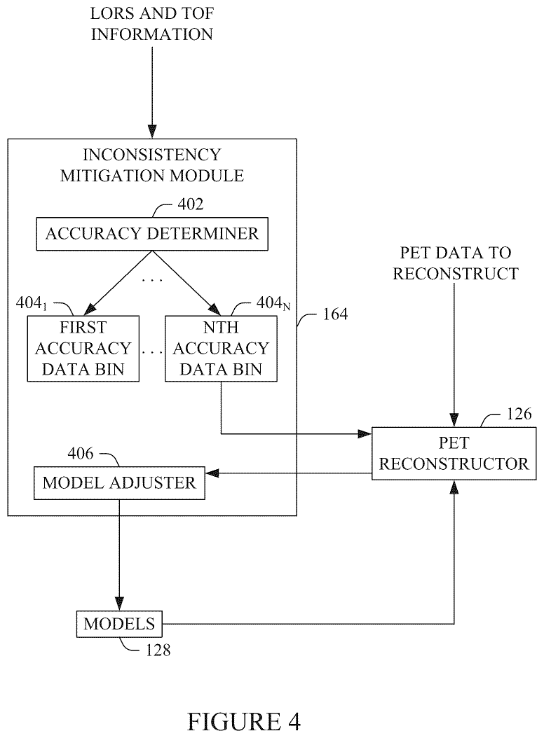

Turning to FIG. 4, an example of the inconsistency mitigation module 164 is schematically illustrated. The inconsistency mitigation module 164 includes an accuracy determiner 402. The accuracy determiner 402 receives, as input, the LORs and TOF information. The accuracy determiner 402 is configured to separate the LORs based on the TOFs, e.g., into two or more bins, such as a first accuracy data bin 404₁, . . . , and an Nth accuracy data bin 404_{N} (collectively referred to herein as bins 404), wherein N is an integer equal to or greater than two. In this example, LORs in the first accuracy data bin 404₁ correspond to lower accuracy TOF LORs, and the LORs in the Nth accuracy data bin 404_{N} correspond to higher accuracy TOF LORs.

In general, only a small fraction of the LORs, e.g., ten to twenty percent (10-20%) will have an effective timing resolution comparable to the timing resolution of Lutetium-based detector crystals. In one instance, the accuracy determiner 402 includes only those 10-20% in the Nth accuracy data bin 404_{N}. In another instance, more or less LORs is included in Nth accuracy data bin 404_{N}. For example, in instances with a large number of counts (high statistics), fewer higher accuracy TOF LORs can be utilized so the criteria can be lower such as seven percent (7%), five percent (5%), four percent (4%), etc. One or more other approaches for sorting the LORs amongst the bins 404 are also contemplated herein.

The higher accuracy TOF LORs from the Nth accuracy data bin 404_{N} are reconstructed using one or more of the models 128 to generate the high-consistent, low-statistic volumetric image data. In one instance, the higher accuracy TOF LORs from the Nth accuracy data bin 404_{N} are reconstructed with the PET reconstructor 126 using one or more of the models 128 to generate the high-consistent, low-statistic volumetric image data. In another instance, the higher accuracy TOF LORs from the Nth accuracy data bin 404_{N} are reconstructed with another reconstructor using one or more of the models 128 to generate the high-consistent, low-statistic volumetric image data.

The inconsistency mitigation module 164 further includes a model adjuster 406. The model adjuster 406 is configured to adjust one or more models of the models 128 based on the high-consistent, low-statistic volumetric image data, updating and/or improving one or more of the models 128. The PET reconstructor 148 employs the updated models 128 to reconstruct PET data, e.g., all of the LORs (e.g., the higher accuracy TOF LORS and the lower accuracy TOF LORS, only the higher accuracy TOF LORS, etc.). Again, the approach described herein improves image quality while maintaining the spatial resolution, relative to a configuration that does not employ the inconsistency mitigation module 164.

In a variation, the higher accuracy TOF LORs from the Nth accuracy data bin 404_{N} are also reconstructed (with the PET reconstructor 126 and/or other reconstructor) without using the models 128 to generate additional volumetric image data. In this variation, the model adjuster 406 is configured to adjust one or more of the models 128 based on the high-consistent, low-statistic volumetric image data and the additional volumetric image data. For example, in one instance, the additional volumetric image data is utilized to facilitate extracting a contour of tissue of interest, such as a contour of an organ. This can be achieved where anatomical data is accurate, not accurate, and/or not utilized. Likewise, the PET reconstructor 126 is used to reconstruct the PET data using the updated models 128.

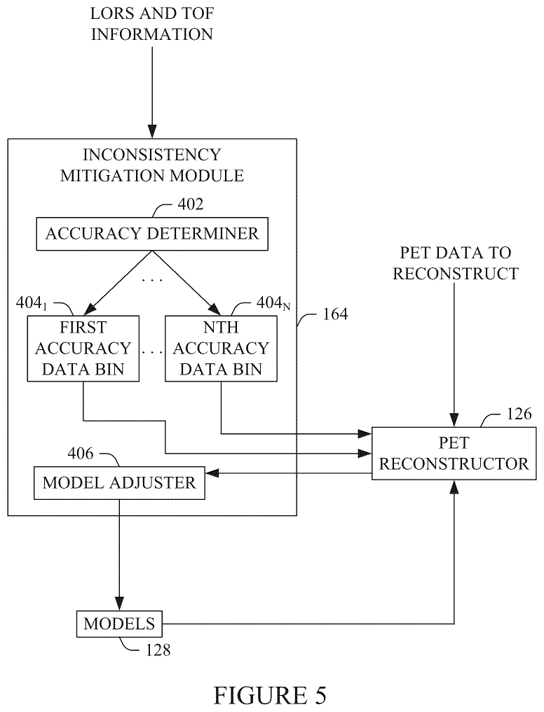

FIG. 5 schematically illustrates another variation of the inconsistency mitigation module 164 described in connection with FIG. 4. Similar to the inconsistency mitigation module 164 described in connection with FIG. 4, the inconsistency mitigation module 164 includes the accuracy determiner 402, which separates the LORs based on the TOFs into, e.g., the first accuracy data bin 404₁, . . . , and the Nth accuracy data bin 404_{N,} where the first accuracy data bin 404₁ correspond to lower accuracy TOF LORs, and the LORs in the Nth accuracy data bin 404_{N} correspond to higher accuracy TOF LORs. Similarly, the higher accuracy TOF LORs from the Nth accuracy data bin 404_{N} are reconstructed using the models 128 to reconstruct high-consistent, low-statistic volumetric image data.

In addition, the lower accuracy TOF LORs are reconstructed using the models 128 with the PET reconstructor 126 and/or other reconstructor to generate other volumetric image data. In this example, the model adjuster 406 adjusts one or more of the models 128 based on the high-consistent, low-statistic volumetric image data and the other volumetric image data, updating and/or improving one or more models 128. For example, in one instance, the two data sets are subtracted, and a difference facilitates identifying region of artifact to improve the models 128. Likewise, the PET reconstructor 126 employs the updated models 128 to reconstruct PET data with image quality improved relative to using the models 128 before the adjustment.

Another variation includes a combination of the examples described in connection with FIG. 4 and FIG. 5. Similar to the inconsistency mitigation module 164 described in connection with FIG. 4, the inconsistency mitigation module 164 includes the accuracy determiner 402, which separates the LORs based on the TOFs into, e.g., the first accuracy data bin 404₁, . . . , and the Nth accuracy data bin 404_{N,} where the first accuracy data bin 404₁ correspond to lower accuracy TOF LORs, and the LORs in the Nth accuracy data bin 404_{N} correspond to higher accuracy TOF LORs. Similarly, the higher accuracy TOF LORs from the Nth accuracy data bin 404_{N} are reconstructed using the models 128 to reconstruct high-consistent, low-statistic volumetric image data.

Similar to the variation described in connection with FIG. 4, the higher accuracy TOF LORs from the Nth accuracy data bin 404_{N} are also reconstructed (with the PET reconstructor 126 and/or other reconstructor) without using the models 128 to generate additional volumetric image data. Similar to the inconsistency mitigation module 164 described in connection with FIG. 5, the lower accuracy TOF LOR from the first accuracy data bin 404₁ are reconstructed using the models 128 with the PET reconstructor 126 and/or other reconstructor to generate other volumetric image data.

In this variation, the model adjuster 406 is configured to adjust one or more of the models 128 based on the high-consistent, low-statistic volumetric image data, the additional volumetric image data, and the other volumetric image data, updating and/or improving one or more models 128. The PET reconstructor 126 employs the updated models 128 to reconstruct PET data, generating volumetric image data with improved image quality and a similar spatial resolution, relative to a configuration that does not employ the inconsistency mitigation module 164.

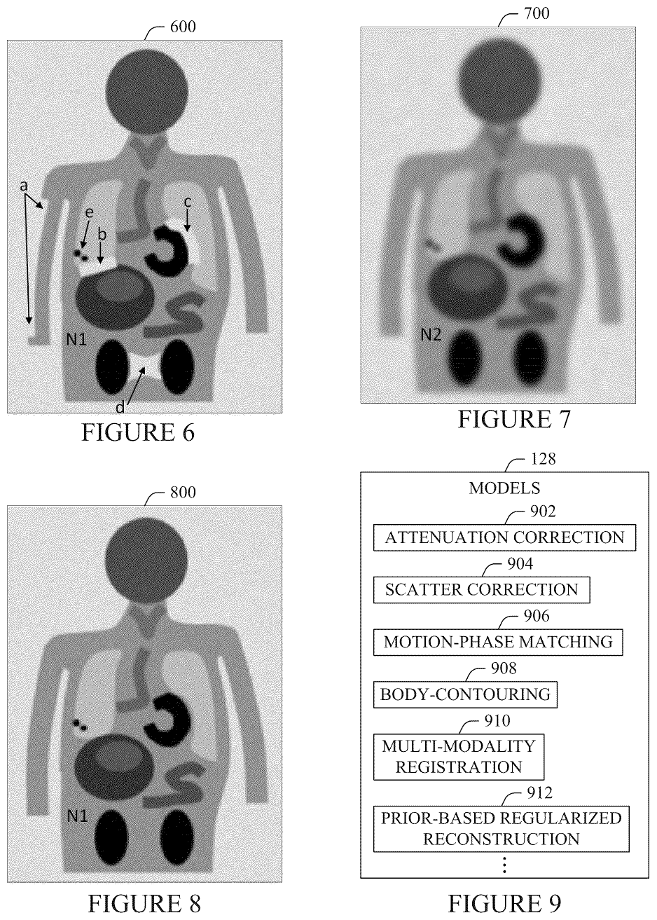

FIGS. 6, 7 and 8 schematically illustrate an example. FIG. 6 schematically illustrates first volumetric image data 600 generated by reconstructing the LORs without employing the inconsistency mitigation module 164 and using the models 128. FIG. 7 schematically illustrates example high-consistent, low-statistic correction volumetric image data 700 generated by reconstructing only the high accuracy TOF data using the models 128. FIG. 8 schematically illustrates final volumetric image data 800 generated by reconstructing some or all of the LORs with the updated models 128.

Initially referring to FIG. 6, in this example, the first volumetric image data 600 is generated by reconstructing the entirety of the acquired PET data using one or more of the models 128. With this example, the anatomical data (e.g., CT, MR, etc.) used by one or more of the models 128 has spatial mismatch regions relative to the PET data. In this example, several image artifact regions are denoted as relatively bright regions (although they may also appear as relatively dark regions in other situations). An inconsistency region “a” is a result of sporadic patient arm movement. An inconsistency region “b” is a result of respiratory motion. An inconsistency region “c” is a result of cardiac motion. An inconsistency region “d” is a result of inaccurate scatter correction between organs with high activity.

Additional types of artifacts and/or image inaccuracies may also occur in various regions due to reconstruction inaccuracies, especially in regions with relatively low activity. The volumetric image data 600 is sensitive to the inconsistency artifacts “a,” “b,” “c,” “d,” etc. at least because of the overall low effective timing resolution (i.e., the higher percentage of lower accuracy TOF LORs relative to higher accuracy TOF LORs). At the same time, the first volumetric image data 600 has higher spatial resolution due to the large number of total counts of the entire acquired PET data. “N1” represents a noise level of the first volumetric image data 600. Regions “e” represent two lesions that are outside of the artifact areas “a,” “b,” “c,” “d,” etc., where the regions “e” are sharp in the first volumetric image data 600 due to the high spatial resolution.

Next at FIG. 7, the high-consistent, low-statistic correction volumetric image data 700 is generated by reconstructing only the high-accuracy TOF LORs (and not the low-accuracy TOF LORs) using the models 128. Since the total counts of the high-accuracy TOF LORs is significantly smaller relative to FIG. 6 where all of the counts (i.e., both the high-accuracy and the low-accuracy TOF LORs) were used, a smoother filtration and/or regularization is applied during the reconstruction to maintain a similar average image noise due to the low-statistics. “N2” represents a noise level of the volumetric image data 700, and “N1” and “N2” are approximately equal in a sense of a standard deviation (SD) over a homogenous region.

Since the high-consistent, low-statistic correction volumetric image data 700 is reconstructed with only the high-accuracy TOF LORs, the high-consistent, low-statistic correction volumetric image data 700 is less sensitive to inconsistency of the artifacts “a,” “b,” “c,” “d,” etc. than the first volumetric image data 600, and the image quality is improved in the low spatial frequencies. In this example, the smoothing results in smearing the regions “e” such that the image quality of the correction volumetric image data 700 is below the image quality of the volumetric image data 600 and, in this instance, a level required for clinical diagnostics.

In one instance, the models 128 dot need high spatial resolution PET data, and the image characteristics of the high-consistent, low-statistic correction volumetric image data 700 are well suited for the update to the models 128. In another instance, the high-accuracy TOF LORs are reconstructed without using the models 128 to generate additional correction volumetric image data (not shown) used to adjust one or more of the models 128, as described herein. In another instance, the low-accuracy TOF LORs are reconstructed using the models 128 to generate other volumetric image data (not shown) that is used to adjust one or more of the models 128, as described herein.

Next at FIG. 8, PET data is reconstructed using the models 128 that are updated with the high-consistent, low-statistic correction volumetric image data 700 or with the high-consistent, low-statistic volumetric image data 700 and the additional volumetric image data and/or the other volumetric image data. In this example, the volumetric image data 800 has at least a same spatial resolution as the volumetric image data 600 with no more image noise than the volumetric image data 600 (“N1”). As such, the regions “e” in the volumetric image data 800 are sharp similarly to the regions “e” in the volumetric image data 600. In addition, the volumetric image data 800 has no or less of the inconsistency artifacts “a,” “b,” “c,” “d,” etc. relative to the volumetric image data 600.

Moving to FIG. 9, an example of the models 128 is schematically illustrated. In this example, the models 128 include an attenuation correction model 902, a scatter correction model 904, a motion-phase matching model 906, a body-contouring model 908, a multi-modality registration model 910, a prior-based regularized reconstruction model 912, and/or one or more other models.

The attenuation correction model 902 employs anatomical data (e.g., CT, MR, etc.) to generate an attenuation correction (μ) map to attenuation-correct the PET data. The coefficients of the map indicate how much the tissues absorb or scatter the gamma photons emitted during the PET scan. Without correction, the PET images would be distorted, leading to inaccurate measurements of tracer concentration. A PET-anatomical data mismatch and/or misregistration can cause falsely low-activity areas in certain regions such as the lung regions. The model adjuster 406 employs the TOF data to update the attenuation correction model 902 to re-shape the attenuation correction map to reduce and/or remove mismatch/misregistration, e.g., so that the anatomical data better matches the PET data. In instances where non-TOF PET data is available, the model adjuster 406 also compares the TOF data and the non-TOF PET data to facilitate detecting regions that should be corrected.

The scatter correction model 904 removes scatter from the PET data. The estimation of scatter is based on initial low resolution PET volumetric image data (which is less sensitive to scatter artifact), and then the scatter estimation and the PET volumetric image data are updated iteratively. The model adjuster 406 employs the TOF data to update the scatter correction model 904 to use the low resolution TOF PET volumetric image data for a more accurate scatter estimation. For scatter correction, the contour of the body is also utilized to differentiate regions of scatter within the body from outside of the body. The model adjuster 406 updates the scatter correction model 904 to utilize the body contour extracted by the updated body-contouring model 908, which is discussed below.

The motion-phase matching model 906 is utilized to correct the PET volumetric image data itself for natural motion of the patient during the acquisition (e.g. respiration, cardiac, etc.). In one instance, several phases or gates are detected, and motion vector fields are estimated to deform (i.e., warped or morphed) the different phases to artificially match a single selected phase. The estimated vector fields are typically regularized to include only low spatial frequencies (in order not to create unnatural structures). In the process of motion phase matching, each phase is reconstructed without having specific corrected attenuation map. The model adjuster 406 updates the motion-phase matching model 906 to provide more accurate results using low-resolution, but artifact free PET images, such as the higher accuracy TOF PET data. Similar to the attenuation-correction model 902, the motion-phase matching model 906 can use the higher accuracy TOF PET data to better match the anatomical data to the PET data.

The body-contouring model 908 is configured to extract a contour of the body, which can be used by the scatter correct model 904, the multi-modality registration model 910, and/or the prior-based regularized reconstruction applying priors to improve the reconstruction itself. The model adjuster 406 updates the body-contouring model 908 to use the high accuracy TOF PET volumetric image data to extract a more accurate contour of the body. For scatter correction, the contour of the body is utilized to different regions of scatter within the body from outside of the body, and the detection of the contour of the body can be extracted with the low-resolution TOF PET volumetric image data, e.g., via a maximum gradient, etc., of a more smoothed profile.

The model adjuster 406 updates multi-modality registration model 910 to use the TOF PET volumetric image data for improving the attenuation-correction model 902 and final PET-CT image registration in the diagnostic image level. Non-TOF PET images sometimes suffer from inaccurate reconstructed activity values in regions with low activity, such as in fatty abdomen regions and in the problem of artifacts outside the body contour, e.g., in breast scans in a prone position. For these types of problems, it may help to enter prior information from the high accuracy TOF data. The model adjuster 406 updates the body prior-to based regularized reconstruction model 912 to use the TOF data to provide prior to where the photons should be distributed within the image. In one instance, the prior-based regularized reconstruction model 912 uses the TOF PET data to add activity to a low activity region, essentially adding back missing information.

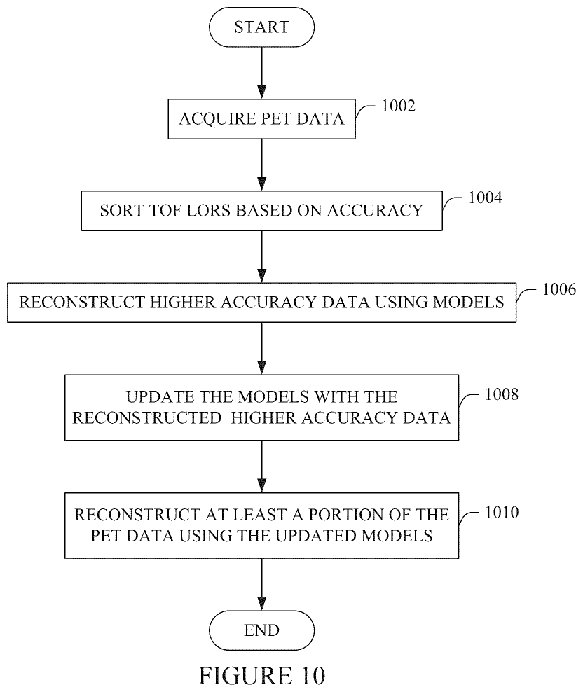

FIG. 10 illustrates a non-limiting example of a flow chart for a computer-implemented method for mitigating reconstruction inconsistencies for a TOF-BGO-PET based reconstruction using only corrected higher accuracy TOF LORs to update the reconstruction models. It is to be appreciated that the ordering of the acts in the method is not limiting. As such, other orderings are contemplated herein. In addition, one or more acts may be omitted, and/or one or more additional acts may be included.

At 1002, a PET scan is performed with a PET imaging with BGO detectors configured to detect Cherenkov photons and TOF reconstruction capabilities, as described herein and/or otherwise. At 1004, the LORs are sorted into multiple groups based on timing resolutions, as described herein and/or otherwise. At 1006, the higher accuracy TOF LORs are reconstructed using the models 128 to generate high-consistent, low-statistic correction volumetric image data, as described herein and/or otherwise.

At 1008, the high-consistent, low-statistic correction volumetric image data is utilized to update the models 128, as described herein and/or otherwise. At 1010, the acquired PET data and/or a sub-set thereof are reconstructed using the updated models 128. The reconstructed data can be displayed, archived, evaluated, etc. Again, the approach described herein improves image quality while maintaining the spatial resolution, relative to a configuration that does not employ the approach described herein.

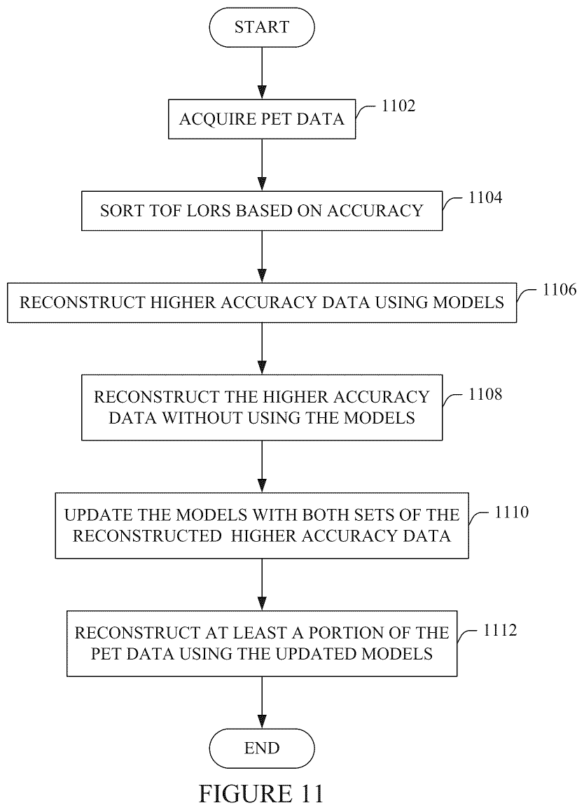

FIG. 11 illustrates a non-limiting example of a flow chart for a computer-implemented method for mitigating reconstruction inconsistencies for a TOF-BGO-PET based reconstruction using corrected and uncorrected higher accuracy TOF LORs to update the reconstruction models. It is to be appreciated that the ordering of the acts in the method is not limiting. As such, other orderings are contemplated herein. In addition, one or more acts may be omitted, and/or one or more additional acts may be included.

At 1102, a PET scan is performed with a PET imaging with BGO detectors configured to detect Cherenkov photons and TOF reconstruction capabilities, as described herein and/or otherwise. At 1104, the LORs are sorted into multiple groups based on timing resolutions, as described herein and/or otherwise. At 1106, the higher accuracy TOF LORs are reconstructed using the models 128 to generate high-consistent, low-statistic correction volumetric image data, as described herein and/or otherwise.

At 1108, the high accuracy TOF PET LORs are also reconstructed without using the models 128 to generate additional correction volumetric image data, as described herein and/or otherwise. At 1110, the high-consistent, low-statistic correction volumetric image data and the additional correction volumetric image data are utilized to update the models 128, as described herein and/or otherwise. At 1112, the acquired PET data and/or a sub-set thereof are reconstructed using the updated models 128. The reconstructed data can be displayed, archived, evaluated, etc.

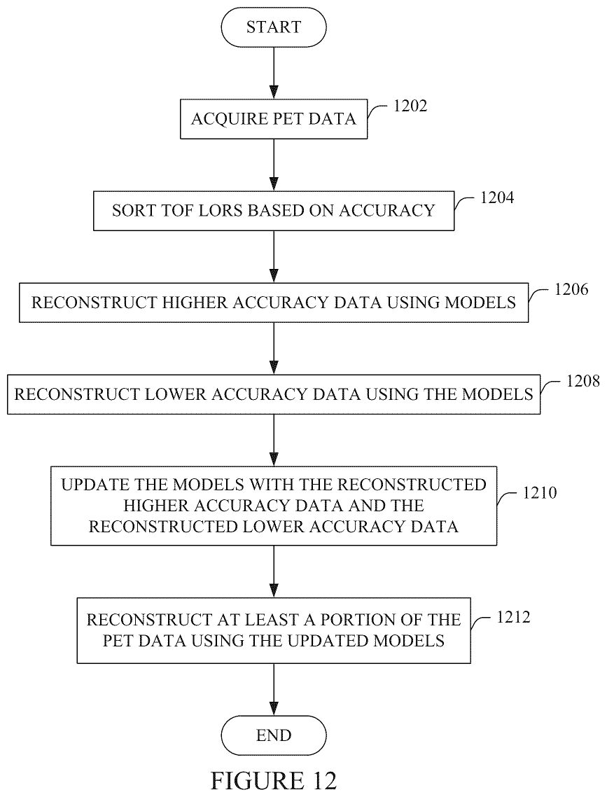

FIG. 12 illustrates a non-limiting example of a flow chart for a computer-implemented method for mitigating reconstruction inconsistencies for a TOF-BGO-PET based reconstruction using corrected higher accuracy TOF LORs and corrected lower accuracy TOF LORs to update the reconstruction models. It is to be appreciated that the ordering of the acts in the method is not limiting. As such, other orderings are contemplated herein. In addition, one or more acts may be omitted, and/or one or more additional acts may be included.

At 1202, a PET scan is performed with a PET imaging with BGO detectors configured to detect Cherenkov photons and TOF reconstruction capabilities, as described herein and/or otherwise. At 1204, the LORs are sorted into multiple groups based on timing resolutions, as described herein and/or otherwise. At 1206, the higher accuracy TOF LORs are reconstructed using the models 128 to generate high-consistent, low-statistic correction volumetric image data, as described herein and/or otherwise.

At 1208, the lower accuracy TOF PET data are reconstructed using the models 128 to generate other volumetric image data, as described herein and/or otherwise. At 1210, the high-consistent, low-statistic correction volumetric image data and the other volumetric image data are utilized to update the models 128, as described herein and/or otherwise. At 1212, the acquired PET data and/or a sub-set thereof are reconstructed using the updated models 128. The reconstructed data can be displayed, archived, evaluated, etc.

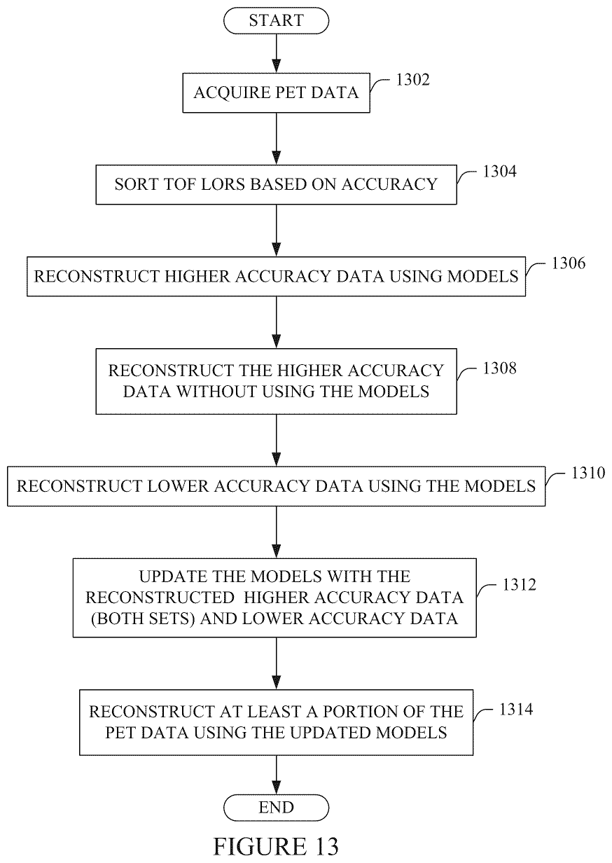

FIG. 13 illustrates a non-limiting example of a flow chart for a computer-implemented method for mitigating reconstruction inconsistencies for a TOF-BGO-PET based reconstruction using corrected higher accuracy TOF LORs, reconstructed uncorrected higher accuracy TOF LORs, and reconstructed corrected lower accuracy TOF LORs to update the reconstruction models. It is to be appreciated that the ordering of the acts in the method is not limiting. As such, other orderings are contemplated herein. In addition, one or more acts may be omitted, and/or one or more additional acts may be included.

At 1302, a PET scan is performed with a PET imaging with BGO detectors configured to detect Cherenkov photons and TOF reconstruction capabilities, as described herein and/or otherwise. At 1304, the LORs are sorted into multiple groups based on timing resolutions, as described herein and/or otherwise. At 1306, the higher accuracy TOF LORs are reconstructed using the models 128 to generate high-consistent, low-statistic correction volumetric image data, as described herein and/or otherwise.

At 1308, the higher accuracy TOF PET LORs are also reconstructed without using the models 128 to generate additional correction volumetric image data, as described herein and/or otherwise. At 1310, the lower accuracy TOF PET data are reconstructed using the models 128 to generate other volumetric image data, as described herein and/or otherwise. At 1312, the high-consistent, low-statistic correction volumetric image data, the additional volumetric image data, and the other volumetric image data are utilized to update the models 128, as described herein and/or otherwise.

At 1314, the acquired PET data and/or a sub-set thereof are reconstructed using the updated models 128. The reconstructed data can be displayed, archived, evaluated, etc. Again, the approach described herein improves image quality while maintaining the spatial resolution, relative to a configuration that does not employ the approach described herein.

The above can be implemented by way of computer readable instructions, encoded, or embedded on the computer readable storage medium, which, when executed by a computer processor, cause the processor to carry out the described acts or functions. Additionally, or alternatively, at least one of the computer readable instructions is carried out by a signal, carrier wave or other transitory medium, which is not computer readable storage medium.

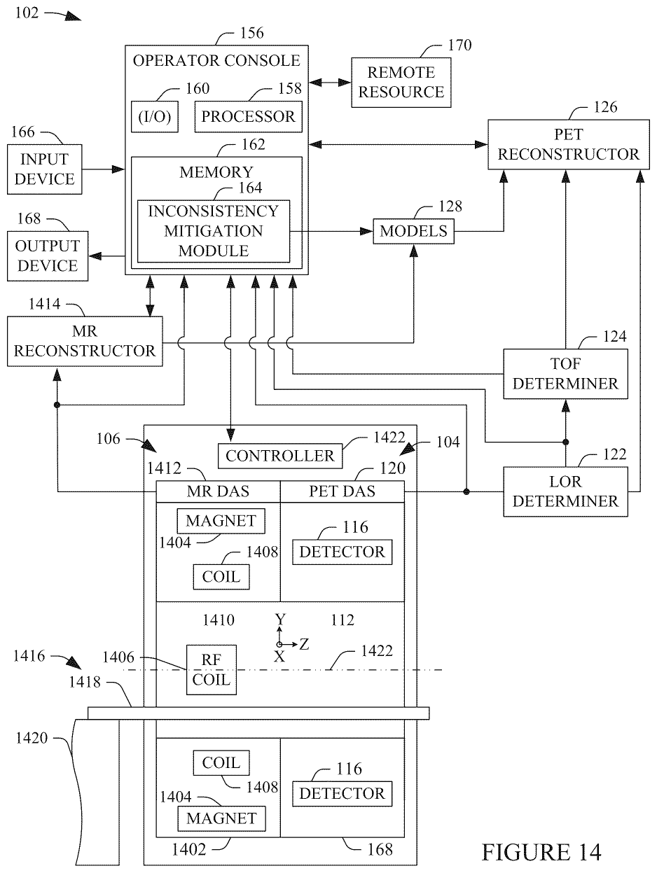

As discussed herein, in general, the imaging system 102 includes the functional imaging sub-system 104 and the anatomical imaging sub-system 106, and, in FIG. 1, the functional imaging sub-system 104 is configured for PET imaging and the anatomical imaging sub-system 106 is configured for CT imaging, where other anatomical imaging sub-systems are contemplated herein. FIG. 14 schematically illustrates an example in which the anatomical imaging sub-system 106 is configured for MR imaging and includes an MR imaging sub-system.

In FIG. 14, the imaging system 102 includes the PET imaging sub-system 104 and an MR imaging sub-system 1402. The MR imaging sub-system 1402 includes a main magnet 1404, a gradient (x, y, and z) coil(s) 1408, and a RF coil 1406. The main magnet 1404 (which can be a superconducting, resistive, permanent, or other type of magnet) produces a substantially homogeneous, temporally constant main magnetic field B0 in an MR bore 1410. The gradient coil(s) 1408 generate time varying gradient magnetic fields along the x, y, and z-axes of the MR bore 1410.

The RF coil 1406 includes a transmit portion that produces radio frequency signals (at the Larmor frequency of nuclei of interest (e.g., hydrogen, etc.)) that excite the nuclei of interest in the MR bore 1410 and a receive portion that detects MR signals emitted by the excited nuclei. In other embodiments, the transmit portion and the receive portion of the RF coil 1406 are located in separate RF coils 1406. A MR data acquisition system (DAS) 1412 processes the MR signals, and a MR reconstructor 1414 reconstructs the data and generates MR images.

A table 1416 includes a cradle 1418 moveably coupled to a frame/base 1420. In one instance, the cradle 1418 is slidably coupled to the frame/base 1420 via a bearing or the like, and a drive system (not visible) including a controller, a motor, a lead screw, and a nut (or other drive system) translates the cradle 1418 along the frame/base 1420 into and out of the MR bore 1410 and/or the PET bore 112. The cradle 1418 is configured to support a subject in the MR bore 1410 and/or PET bore 112 for loading, scanning, and/or unloading the subject.

A controller 1422 is configured to control components of the MR imaging sub-system 1402 such as the main magnet 1404, the gradient coil(s) 1408, the RF coil 1406, an operation of the table 1416, etc. For example, in one embodiment the controller 1422 includes a table controller configured to control motion and/or height of the table 1416 for loading, scanning and/or unloading the subject or object. Where the PET and MR sub-systems 104 and 106 are separate imaging systems, each can have its own controller.

The PET bore 112 and the MR bore 1410 are disposed along a common longitudinal or z-axis 1422. The operator console 156 is substantially similar to that described in connection with FIG. 1, except the imaging sub-system 106 is configured for the MR imaging instead of the CT imaging. As such, is not described in detail again. In instances in which the imaging sub-systems 104 and 106 are separate imaging systems, each of the sub-systems 104 and 106 will have its own controller, table, and operator console.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include such additional elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”. The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present disclosure. That is, features of the described embodiments can be combined with any appropriate aspect described above and optional features of any one aspect can be combined with any other appropriate aspects. Similarly, features set forth in dependent claims can be combined with non-mutually exclusive features of other dependent claims, particularly where the dependent claims depend on the same independent claim. Single claim dependencies may have been used as practice in some jurisdictions that require them, but this should not be taken to mean that the features in the dependent claims are mutually exclusive.

What is claimed is:

1. A PET imaging system, comprising:
Bismuth Germanate (BGO) crystals that generate Cherenkov photons in response to excitation by photons from positron annihilation events and photosensors configured to detect the Cherenkov photons and generate signals indicative thereof;
a line of response (LOR) determiner configured to identify, based on the signals, coincident photon pairs along each LOR;
a time-of-flight (TOF) determiner configured to determine a timing resolution for each of the coincident photon pair along each LOR;
an accuracy determiner configured to bin the LORs, based on the timing resolutions, into at least higher accuracy TOF LORs and lower accuracy TOF LORs;
a reconstructor configured to reconstruct the higher accuracy TOF LORs using models and generate volumetric image data; and
a model adjuster configured to adjust at least one model of the models based on the volumetric image data,
wherein the reconstructor is configured to reconstruct at least a subset of the LORs using the updated models.

2. The PET imaging system of claim 1, wherein the reconstructor is further configured to reconstruct the higher accuracy TOF LORs without using the models and generate additional correction data, and the model adjuster further configured to adjust the at least one model of the models based on the additional correction data.

3. The PET imaging system of claim 1, wherein the reconstructor is further configured to reconstruct the lower accuracy TOF LORs using the models and generate other correction data, and the model adjuster further configured to adjust the at least one model of the models based on the other correction data.

4. The PET imaging system of claim 1, wherein the reconstructor is further configured to reconstruct the higher accuracy TOF LORs without using the models and generate additional correction data, reconstruct the lower accuracy TOF LORs using the models and generate other correction data, and the model adjuster further configured to adjust the at least one model of the models based on the additional correction data and the other correction data.

5. The PET imaging system of claim 1, wherein the models include at least one of an attenuation correction, a scatter correction, a motion-phase matching, a body-contouring, a multi-modality registration, and a prior-based regularized reconstruction.

6. The PET imaging system of claim 1, wherein reconstructed volumetric image data using the models has a first image quality, reconstructed volumetric image data using the updated models has a second image quality, and the second image quality is greater than the first image quality.

7. The PET imaging system of claim 6, wherein the reconstructed volumetric image data using the models has a first spatial resolution, the reconstructed volumetric image data using the updated models has a second spatial resolution, and the second spatial resolution is at least the same as the first spatial resolution.

8. A computer-implemented method, comprising:
receiving signals from photosensors of a PET imaging system, wherein the signals are indicative of Cherenkov photons produced by BGO crystals in response to excitation by photons from positron annihilation events directed in substantially opposite directions along LORs;
identifying, based on the signals, coincident photons along each LOR;
determining a timing resolution for each identified pair of coincident photons;
binning the LORs, based on the timing resolutions, into at least higher accuracy TOF LORs and lower accuracy TOF LORs;
reconstructing the higher accuracy TOF LORs using models to generate volumetric image;
adjusting at least one model of the models based on the volumetric image; and
reconstructing at least a subset of the LORs using the updated models.

9. The computer-implemented method of claim 8, further comprising:
reconstructing the higher accuracy TOF LORs without using the models to generate additional correction data; and
adjusting the at least one model of the models based on the correction data and the additional correction data.

10. The computer-implemented method of claim 8, further comprising:
reconstructing the lower accuracy TOF LORs using the models to generate other correction data; and
adjusting the at least one model of the models based on the correction data and the other correction data.

11. The computer-implemented method of claim 8, further comprising:
reconstructing the higher accuracy TOF LORs without using the models to generate additional correction data; and
reconstructing the lower accuracy TOF LORs using the models to generate other correction data; and
adjusting the at least one model of the models based on the correction data, the additional correction data, and the other correction data.

12. The computer-implemented method of claim 8, wherein the models include at least one of an attenuation correction, a scatter correction, a motion-phase matching, a body-contouring, a multi-modality registration, and a prior-based regularized reconstruction.

13. The computer-implemented method of claim 8, wherein reconstructed volumetric image data using the models has a first image quality, reconstructed volumetric image data using the updated models has a second image quality, and the second image quality is greater than the first image quality.

14. The computer-implemented method of claim 13, wherein the reconstructed volumetric image data using the models has a first spatial resolution, the reconstructed volumetric image data using the updated models has a second spatial resolution, and the second spatial resolution is at least the same as the first spatial resolution.

15. A computer readable storage medium encoded with computer executable instructions, which when executed by a processor, causes the processor to:
receive signals from photosensors of a PET imaging system, wherein the signals are indicative of Cherenkov photons produced by BGO crystals in response to excitation by photons from positron annihilation events directed in substantially opposite directions along LORs;
identify, based on the signals, coincident photons along each LOR;
determine a timing resolution for each identified pair of coincident photons;
bin the LORs, based on the timing resolutions, into at least higher accuracy TOF LORs and lower accuracy TOF LORs;
reconstruct the higher accuracy TOF LORs using models to generate volumetric image;
adjust at least one model of the models based on the volumetric image; and
reconstruct at least a subset of the LORs using the updated models.

16. The computer readable storage medium of claim 15, wherein the instructions further cause the processor to:
reconstruct the higher accuracy TOF LORs without using the models to generate additional correction data; and
adjust the at least one model of the models based on the correction data and the additional correction data.

17. The computer readable storage medium of claim 15, wherein the instructions further cause the processor to:
reconstruct the lower accuracy TOF LORs using the models to generate other correction data; and
adjust the at least one model of the models based on the correction data and the other correction data.

18. The computer readable storage medium of claim 15, wherein the instructions further cause the processor to:
reconstruct the higher accuracy TOF LORs without using the models to generate additional correction data;
reconstruct the lower accuracy TOF LORs using the models to generate other correction data; and
adjust the at least one model of the models based on the correction data, the additional correction data, and the other correction data.

19. The computer readable storage medium of claim 15, wherein reconstructed volumetric image data using the models has a first image quality, reconstructed volumetric image data using the updated models has a second image quality, and the second image quality is greater than the first image quality, and the second spatial resolution is at least the same as the first spatial resolution.

20. The computer readable storage medium of claim 19, wherein reconstructed volumetric image data using the models has a first spatial resolution, reconstructed volumetric image data using the updated models has a second spatial resolution, and the second spatial resolution is at least the same as the first spatial resolution.
